(19) United States
(12) Patent Application Publication
(10) Pub. No.: US 2010/0239504 A1
(43) Pub. Date: Sep. 23, 2010

(54) IMAGING NUCLEIC ACID BINDING PROTEINS

(75) Inventors: Philip Kuocherng Liu, Winchester, MA (US); Christina Huang Liu, Winchester, MA (US)

Correspondence Address: OCCHIUTI ROHLICEK & TSAO, LLP, 10 FAWCETT STREET, CAMBRIDGE, MA, 02138, US

(73) Assignee: The General Hospital Corporation, Boston, MA (US)

(21) Appl. No.: 12/669,413

(22) PCT Filed: Jul. 17, 2008
(86) PCT No.: PCT/US08/70272

§ 371 Date: May 24, 2010

Related U.S. Application Data
(60) Provisional application No. 60/959,878, filed on Jul. 17, 2007.

(51) Int. Cl. A61K 49/12; A61K 31/7088; C07H 21/04

(52) U.S. Cl. 424/9.35; 514/44 R; 536/23.1; 428/402

(57) ABSTRACT

Reporter conjugates for non-invasive in vivo detection, e.g., imaging, of expression or activity of nucleic acid binding proteins are disclosed. The conjugates include a targeting nucleic acid linked to a reporter group, e.g., a contrast agent, such as a paramagnetic label that can be used with magnetic resonance imaging. The targeting nucleic acid can be a double-stranded nucleic acid that binds to the nucleic acid binding protein the expression of which is to be imaged. In some embodiments, the contrast agent is a chelated metal such as gadolinium or dysprosium. The invention also features methods to image protein expression in various tissues, including the brain, and therapeutic methods.

### RELATED APPLICATION

This application claims priority to U.S. provisional Application No. 60/959,878, filed on Jul. 17, 2007, the content of which is incorporated by reference in its entirety.

### GOVERNMENT SUPPORT

The inventions described and claimed herein were made with government support under R01NS045845, R21NS057556, and R21DA024235 (awarded by N111). The government has certain rights in this application.

### TECHNICAL FIELD

This invention relates to detecting, e.g., imaging, nucleic acid binding proteins in various tissues using, e.g., magnetic resonance (MR) imaging, and more particularly to imaging of nucleic acid binding proteins in the brain.

### BACKGROUND

A number of different approaches to imaging cells have been investigated using either optical, e.g., using green fluorescent protein, bioluminescence, or near infrared fluorescence, or nuclear imaging techniques. Common limitations to these techniques are limited penetration depth (optical techniques) or spatial resolution (nuclear techniques). Recent advances in magnetic resonance (MR) imaging and in particular MR microscopy have led to improved image resolution. However, compared to optical and nuclear techniques, molecular probe detection by MR is several magnitudes less sensitive. On the other hand, MR imaging offers much improved spatial resolution with anatomical precision compared to other modalities such as optical imaging, computer tomography (CT), and positron emission tomography (PET).

In all of these imaging modalities, the common goal is to deliver a suitable contrast agent or label to the relevant tissue, and more specifically into the cells. In the brain, for example, one must typically find a way to overcome the blood-brain-barrier. In addition, most of the known contrast agents, for example, for MR imaging, have limited permeability to cells when administered to live subjects, and as a result the limited permeability provides only a short and often unstable window for MR imaging.

### SUMMARY

The invention is based, in part, on the discovery that short nucleic acid sequences, e.g., phosphorothioated nucleic acid sequences, linked to one or more reporter groups to form reporter conjugates, can enter cells without the need for translocation sequences or receptor, and enable the detection of nucleic acid binding proteins. By properly designing the nucleic acid to bind specifically to a nucleic acid binding protein (such as a transcriptional regulator) in a cell, the new reporter conjugates can be used to detect, e.g., image, expression of target nucleic acid binding proteins non-invasively in a variety of living cells or tissues, such as tissues of the brain, liver, pancreas, heart, lung, spinal cord, prostate, breast, gastrointestinal tract, ovary, skin, and kidney.

The reporter group can be an MR contrast agent, such as a paramagnetic label, e.g., a superparamagnetic iron oxide particle with a maximum diameter between about 1 nm and 2000 nm, e.g., between about 2 nm and 1000 nm. In some embodiments, the maximum particle diameter is between 10 nm and 500 nm (e.g., between about 10 nm and 200 nm, between about 20 nm and 500 nm, and between about 20 nm and 200 nm). The particle can be attached to the targeting nucleic acid, e.g., through entrapment in a cross-linked dextran. In some embodiments, the paramagnetic label is a chelated metal such as Gd³⁺ or Dy³⁺.

The reporter group can also be a fluorescent label, e.g., a FITC, Texas Red, Rhodamine, or a near-infrared fluorophore (e.g., indocyanine green (ICG). Cy3 5.5, or a quantum dot). In other embodiments, the reporter group is or includes a radionuclide. e.g., ¹¹C, ¹³N, ¹⁵O, or ¹⁸F.

In one aspect, the invention features reporter conjugates for imaging nucleic acid binding proteins that include a single targeting nucleic acid (e.g., a double-stranded nucleic acid) linked to one or more reporter groups. These targeting nucleic acids and reporter groups are described in detail herein.

In another aspect, the invention features methods of detecting, e.g., imaging, a nucleic acid binding protein in a tissue in vivo. The methods include obtaining a reporter conjugate including a targeting nucleic acid linked to a reporter group, wherein the targeting nucleic acid hinds specifically to a target nucleic acid protein corresponding to the nucleic acid binding protein to be imaged; administering the reporter conjugate to the tissue in an amount sufficient to provide a detectable image; allowing sufficient time to pass to allow a sufficient amount of unbound reporter conjugate (e.g., a majority of unbound conjugate) to leave the tissue; and imaging the tissue, wherein a detectable image of the reporter group in the tissue indicates the presence of the nucleic acid binding protein. The target nucleic acid binding protein can be a therapeutic protein previously delivered to the tissue. The tissue can be, e.g., brain, heart, lung, liver, pancreas, spinal cord, prostate, breast, gastrointestinal system, ovary, or kidney tissue. The tissue can be within a patient. e.g., a human patient. The reporter group can be a superparamagnetic iron oxide particle with a maximum diameter between about 1 nm and 2000 nm. The reporter conjugate can be administered by, e.g., intravenous injection or intra-cerebroventricular infusion.

In another aspect, the invention features reporter conjugates for imaging nucleic acid binding proteins that include a single targeting nucleic acid linked to one or more superparamagnetic iron oxide particles with a maximum diameter between about 1 nm and 1000 nm (e.g., between about 10 and 100 nm). In some embodiments, the particles include a monocrystalline iron oxide nanoparticle (MION), superparamagnetic iron oxide nanoparticle (SPION), ultra small superparamagnetic iron oxide particle (USPIO), or a cross-linked iron oxide (CLIO) particle. The particle can be surrounded by a polymeric coating material, e.g., cross-linked dextran, carboxymethylated dextran, carboxydextran, starch, polyethylene glycol, arabinogalactan, glycosaminoglycan, organic siloxane, or sulfonated styrenedivinylbenzene, to aid in coupling of the nanoparticle to other moieties.

In an example, the reporter conjugate consists essentially of a single targeting nucleic acid linked to one or more paramagnetic iron oxide particles. In another example, the nucleic acid is linked to the particles via a bridge agent (e.g. biotin or avidin) that is covalently linked to the nucleic acid or the particles. The invention also features a composition containing a plurality of the above described reporter conjugates where each of the reporter conjugates contains only one targeting nucleic acid that is linked to one or more paramagnetic iron oxide particles. The maximum diameter of the particles can be between 1 nm and 1000 nm.

In another aspect, the invention features methods of imaging target cells, e.g., neurons (e.g., cornu ammonis neurons) that are undergoing or have undergone programmed cell death in a tissue. The methods include obtaining a reporter conjugate including a targeting nucleic acid linked to a reporter group, wherein the targeting nucleic acid binds specifically to a target nucleic acid binding protein corresponding to the target cells; administering the reporter conjugate to the tissue in an amount sufficient to provide a detectable image; allowing sufficient time to pass to allow a sufficient amount of unbound reporter conjugate (e.g., a majority of unbound conjugate) to leave the tissue; and imaging the tissue, wherein a presence of a detectable image of the reporter group in the tissue indicates that the cells in the tissue have not undergone programmed cell death, and an absence of a detectable image of the reporter group indicates that the cells are undergoing or have undergone programmed cell death.

In another aspect, the invention features methods of treating a disorder, e.g., a cancer, in a patient. The methods include obtaining a conjugate including a targeting nucleic acid linked to a therapeutic agent and a reporter group, wherein the targeting nucleic acid binds specifically to a target nucleic acid binding protein corresponding to a target organ or tissue; and administering the conjugate to a patient in an amount sufficient to treat the disorder. In some embodiments, the targeting nucleic acid preferentially binds to an oncoprotein.

In another aspect, the invention features the use of a reporter conjugate including a targeting nucleic acid linked to a reporter group, wherein the targeting nucleic acid binds specifically to a target cellular nucleic acid binding protein, in the preparation of a pharmaceutical composition for imaging a cellular nucleic acid binding protein in a tissue in vivo

In another aspect, the invention features methods of detecting. e.g., imaging, expression or activity (e.g., a nucleic acid binding activity) of a target nucleic acid binding protein in a tissue in vivo, by obtaining a reporter conjugate including a targeting nucleic acid linked to a reporter group, wherein the targeting nucleic acid binds specifically to a target nucleic acid binding protein corresponding to the target protein the expression or activity of which is to be imaged; administering the reporter conjugate to the tissue in an amount sufficient to provide a detectable image; allowing sufficient time to pass to allow a sufficient amount of unbound reporter conjugate (e.g., a majority of unbound conjugate) to leave the tissue; and imaging the tissue, wherein a detectable image of the reporter group in the tissue indicates expression or activity of the target protein.

In other aspects, the invention features methods of imaging a nucleic acid binding protein in a tissue by obtaining a reporter conjugate including a targeting nucleic acid linked to a reporter group, wherein the targeting nucleic acid hinds specifically to a target nucleic acid binding protein corresponding to the nucleic acid binding protein to be imaged; administering the reporter conjugate to the tissue in an amount sufficient to provide a detectable image; allowing sufficient time to pass to allow a sufficient amount of unbound reporter conjugate (e.g., a majority of unbound conjugate) to leave the tissue; and imaging the tissue, wherein a detectable image of the reporter group in the tissue indicates the presence of the nucleic acid binding protein.

The invention also includes methods of treating a cancer cell in a patient by obtaining a conjugate including a targeting nucleic acid linked to an anti-cancer agent, wherein the targeting nucleic acid binds specifically to a target nucleic acid binding protein corresponding to (e.g., expressed by) the cancer cell; and administering the conjugate to the patient in an amount sufficient to inhibit growth of the cancer cell. The conjugate can further include a reporter group.

The invention also includes methods of treating a disorder in a patient by obtaining a conjugate including a targeting nucleic acid linked to a therapeutic agent, e.g., a dextran-coated therapeutic agent, wherein the targeting nucleic acid binds specifically to a target nucleic acid binding protein corresponding to (e.g., expressed by) a desired target organ or tissue, and administering the conjugate to the patient in an amount sufficient to treat the disorder. The conjugate can further include a reporter group.

In other embodiments, the invention includes methods of decreasing activity (e.g., transcription activating or transcription repressing activity) of a nucleic acid binding protein in a cell and, optionally, imaging a nucleic acid binding protein by obtaining a reporter conjugate including a nucleic acid, e.g., a phosphorothioated nucleic acid (e.g. a phosphorothioated DNA), that hinds specifically to a target nucleic acid protein, and administering the conjugate to a cell in an amount sufficient to inhibit activity of the target nucleic acid binding protein (e.g., by competing for binding of the target nucleic acid binding protein to its endogenous target), and, optionally, allowing sufficient time to pass to allow a sufficient amount of unbound reporter conjugate (e.g., a majority of unbound conjugate) to leave the tissue and imaging the tissue.

The invention also includes methods of imaging (e.g. visualizing or locating) a cell type that expresses a nucleic acid binding protein in a subject. The methods include obtaining a conjugate including a targeting nucleic acid linked to a reporter group, wherein the targeting nucleic acid binds specifically to a target nucleic acid binding protein that is expressed by the cell type to be imaged, administering the conjugate to a subject in an amount sufficient to produce a detectable image, and imaging the tissue, wherein the presence of the conjugate is indicative of the cell type. The cell type to be imaged can be, e.g., a cancer cell, a transgenic cell, or a stem cell (e.g., an embryonic stem cell).

In another embodiment, the invention includes use of a reporter conjugate including a targeting nucleic acid linked to a reporter group, wherein the targeting nucleic acid binds specifically to a target nucleic acid binding protein, in the preparation of a pharmaceutical composition for imaging a nucleic acid binding protein in a tissue in vivo. The reporter conjugate can further include a therapeutic agent.

A nucleic acid that binds “specifically” to a target nucleic acid binding protein binds preferentially to the target, and does not substantially bind to other molecules or compounds in a biological sample.

As used herein, “paramagnetic” means having positive magnetic susceptibility and lacking magnetic hysteresis (ferromagnetism).

As used herein, “superparamagnetic” means having positive magnetic susceptibility and lacking magnetic hysteresis (ferromagnetism) at temperatures below the Curie or the Néel temperature of the material.

As used herein, an “oncoprotein” is an allelic form of a protein that is associated with increased risk of cancer, e.g., a mutant form of a proto-oncogene or tumor suppressor protein, or a viral oncoprotein. Numerous examples of oncoproteins are known in the art (see, e.g. Vogelstein and Kinzler, Nat. Med., 10:789-99 (2004)).

As used herein, a disorder or injury mediated by a nucleic acid binding protein is one that is associated, linked, connected, related, or directly or indirectly caused by expression or activity (e.g., increased or abnormal activity) of the nucleic acid binding protein.

The new conjugates and methods allow real time non-invasive imaging of nucleic acid binding protein expression and/or activity. e.g., by methods such as MRI, and avoid the need for biopsies. The imaging is safe and can be performed as often as is needed over a period of several days.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A to 1H are a series of schematic representations of reporter conjugates, showing certain possible attachments of reporter groups, such as contrast agents or labels, that can be, linked, e.g., via covalent bonds, directly or indirectly to one (FIGS. 1A-1D) or both ends (FIGS. 1E to 1H) of a double- or single-stranded nucleic acid, or with additional sites within the targeting nucleic acids. Reporter groups, e.g., contrast agents and labels that can be used include, but are not limited to, paramagnetic agents, fluorescent labels (FITC, rhodamine, Texas Red), radioactive isotopes, individually or combinations.

FIG. 1I is a legend depicting the symbols used in FIGS. 1A to 1H.

FIGS. 2A and 2B are two schematic representations of reporter conjugates that can be used for therapeutic purposes. e.g. to treat cancer, when the cancer cells contain a known target nucleic acid binding protein.

FIG. 2C is a legend depicting the symbols used in FIGS. 2A and 2B.

FIGS. 3A and 3B are R2* maps showing transcripts of c-Fos (FIG. 3A) and actin (FIG. 3B) in ischemic and sham-operated animals.

FIGS. 3C and 3D are subtraction maps of the R2* maps in FIGS. 3A and 3B, respectively.

FIG. 3E are R2* subtraction maps depicting elevation of AP-1 binding protein in the brain of ischemic animals as compared to sham-operated animals.

FIG. 4 is a chart depicting R2* values representing AP-1 nucleic acid binding protein in the somatosensory cortices in sham-operated (Sham) and cerebral ischemia-induced (Ischemia) live animals.

FIG. 5A are caudal views of R2* maps of SPION-AP1ds infused live brains after acute amphetamine exposure (top two rows) or saline control (bottom row).

FIG. 5B are a series of schematic representation outlining regions of interest (ROI) for statistic analyses performed according to Paxino's Brain Atlas.

FIG. 5C is a chart showing results of quantitative analyses in ROI from R2* maps of mice that were administered with amphetamine or saline. Brain regions with significant elevation in AP-1 activities include medial pre-frontal cortex (mPFC), nucleus accumbens (NAc), caudate putamen (CPu), all of which are part of the dopaminergic pathway, and somatosensory cortex (SSC).

FIG. 5D is a chart showing results of quantitative analyses in ROI from R2* maps from mice that were pre-treated with SCH23390 or saline before Amph administration. CPu exhibited significant reduction in AP-1 activity in SCH23390 pre-treated mice than SAL pre-treated mice.

FIG. 6A is a picture of caudal views of R2* maps of SPION-fosB infused live brains after amphetamine exposure (bottom row) compared to saline control (top row).

FIG. 6B is a chart showing FosB mRNA elevation after acute amphetamine stimulation in different areas of brains

FIG. 7A is a picture of caudal views of R2* maps of SPION-fosB infused live brains, showing depicted hotspots having reduced fosB mRNA in mPFC, NAc, and CPu.

FIG. 7B is a chart of ROI analyses result showing significant fosB mRNA reduction in SCH123390 pre-treated mice as compared with that in SAL pre-treated mice.

### DETAILED DESCRIPTION

The invention relates to new methods and compositions for detecting (e.g., imaging) the expression and/or activity of specific target nucleic acid binding proteins, in in various cells and tissues, such as the brain, non-invasively using various imaging modalities, such as MR imaging.

### General Methodology

The new imaging methods use reporter conjugates to detect, e.g., image, the uptake and distribution of conjugated targeting nucleic acids, e.g., oligodeoxyribonucleotides (ODN), delivered to the brain or other tissues in live animals and humans. The conjugates include a reporter group, such as a contrast agent or a label, e.g., an MR contrast agent. e.g., iron oxide nanoparticles (e.g., MION-dextran) linked to a targeting nucleic acid (such as a double-stranded ODN) that binds specifically to a particular target nucleic acid binding protein. The conjugate is delivered to the tissue containing, or thought to contain, a target nucleic acid binding protein, of which the distribution, expression, and/or activity is to be imaged. For example, if the reporter conjugate is to be delivered to the brain, one can use convection-enhanced delivery to the cerebral ventricles such as to the lateral ventricle (Liu et al., Ann Neurol., 36:566-76, 1994; and Cui et al. J. Neurosci., 19:1335-44, 1999) or the 4^{th} ventricles (Sandberg et al., J. Neuro-Oncology, 58:187-192, 2002). Delivery can also be intrathecal (Lin et al., Magn. Reson. Med., 51:978-87, 2004) or by any additional routes that lead directly or indirectly to brain cells. The general methodology of similar methods is described in detail in WO 2006/023888.

After a sufficient amount of time (e.g., 15 or 30 minutes, or 1, 2, 3, 4, 5, 6, 7, 8 10, 12, 15, 18, 24, 30, 36, or 48 hours) for the reporter conjugate to be localized to, and internalized by, the appropriate cells within the tissue, and for sufficient unbound conjugates (e.g. a majority of the unbound conjugates) to leave the tissue, the tissue is imaged. For example, the tissue can be imaged with a series of high-resolution T2*-weighted MR images, e.g., taken 1, 2, or 3 days after infusion of the reporter conjugate.

To use the new conjugates and methods to detect protein expression or activity, the targeting nucleic acid can be prepared as a sequence that is designed to bind specifically to the target nucleic acid binding protein (e.g., a consensus nucleic acid binding sequence). Thus, if a reporter conjugate including this sequence is detected in cells in a tissue, it provides a clear indication that the target nucleic acid binding protein is present in the cell, and thus that the target nucleic acid binding protein is expressed and/or active.

The new reporter conjugates and imaging methods open a new route to detect and track the delivery and uptake of nucleic acid molecules that encode nucleic acid binding proteins in live animals for neuroscience research and various clinical applications.

### Reporter Conjugates

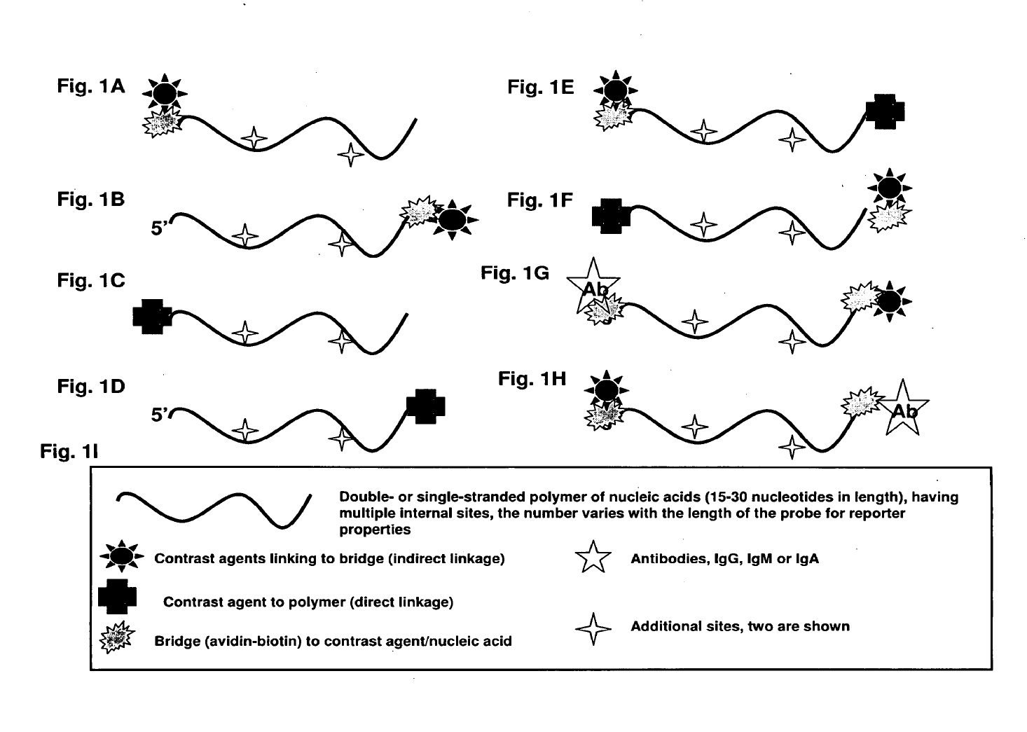

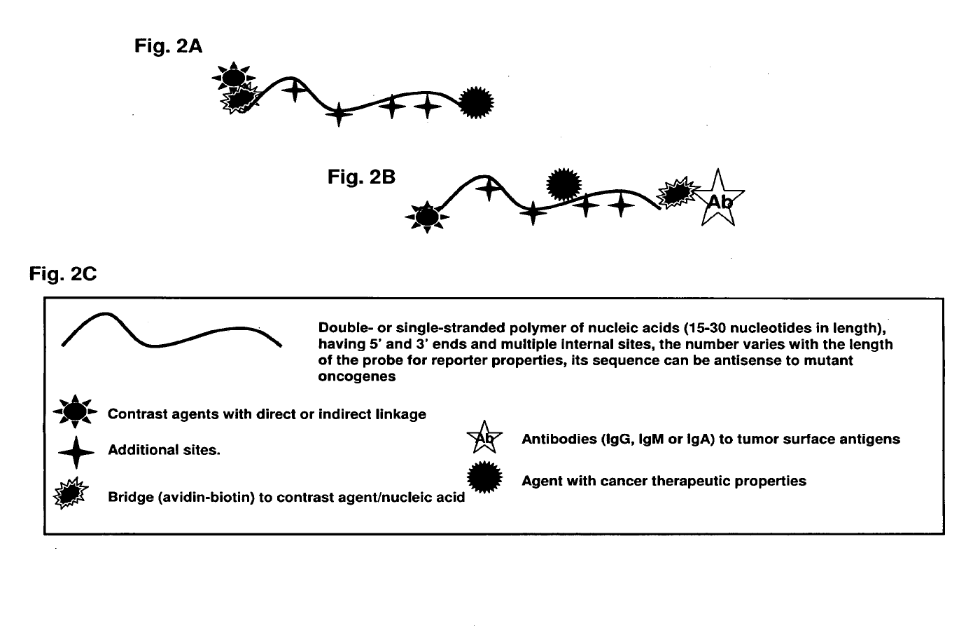

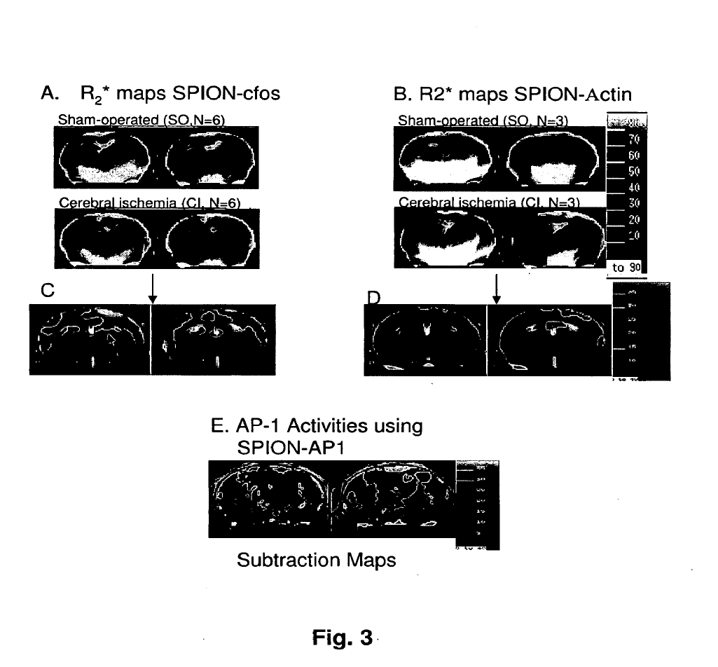

The reporter conjugates are prepared by conjugating or linking one or more targeting nucleic acids to one or more reporter groups, such as magnetic particles that change the relaxivity of the cells once internalized so that they can be imaged using MR. One targeting nucleic acid can have multiple (e.g., 2, 3, or more) reporter groups attached (all or some the same or different), or a set of numerous reporter conjugates can be created in which they all have the same targeting nucleic acid and 2 or more different reporter groups within the set. Several variations of the different types of reporter conjugates are shown in FIGS. 1A to 1I.

A general rationale is that each reporter conjugate must contain a sequence capable of binding to a nucleic acid binding protein, and the conjugate must also be able to form complex with the protein for a period of time long enough to image transient conjugate retention. Moreover, the does of the conjugate must be high enough to generate sufficient contrast-to-noise ratio and low enough to be cleared from a target within a reasonable span of time. Because targeting and reporting a nucleic acid binding protein are based on specific binding of the nucleic acid in the conjugate to its target, the conjugate must have sufficient reporting sensitivity. For example, the conjugate has sufficient reporting sensitivity when its loading capacity is one, that is, one targeting nucleic acid to one contrast agent. In the case of more than one contrast agents per nucleic acid, the sensitivity will be even higher. In contrast, four nucleic acids per contrast agent (loading capacity of 4, as seen in conventional MRI imaging) will reduce reporting sensitivity by 75%. Due to the reporting sensitivity, the conjugate described therein allows one to obtain unexpectedly specific and strong signals.

As shown in FIGS. 1A and 1B, the conjugate includes a targeting double- or single-stranded nucleic acid of 15 to 30 nucleotides (also referred to herein as an oligonucleotide or ODN), one or more reporter groups, such as a contrast agent, linked to either the 5′ or 3′ ends of one or both strands of the ODN, either directly, e.g. by a covalent bond or via an optional linker group or “bridge” (e.g., a linkage of a desired length) between the ODN and the reporter group(s). The targeting nucleic acid can be, e.g., either single- or double-stranded DNA or RNA. The ODN may include one or multiple internal sites that can be attached to a reporter group, e.g., labeled, for example, with a radioactive or fluorescent label. More than 50 unique reporter groups can be made in ˜2 kilobases of a polynucleotide. For example, 50 different reporter conjugates can be made that specifically bind to a specific target nucleic acid binding protein, e.g., to different portions of the same target. All 50 conjugates can have the same or different reporter groups, and could have different (e.g., up to 50 different) reporter groups on the 50 different conjugates. This can be used to provide signal amplification.

FIGS. 1E to 1H show reporter conjugates that include two or more reporter groups, as well as an optional antibody that can be attached at either end of the molecule (FIGS. 1G and 1H). These antibodies can typically be ones that hind specifically to cell-surface antigens of particular cells or cell types to direct the reporter conjugate to the appropriate cells. Once on the surface of the cell, the reporter conjugates pass through the cell membrane and into the cells, thereby delivering the reporter group into the cell. Once in the cell, the targeting nucleic acids hind preferentially to their specific target nucleic acid binding proteins and remain bound within the cell. Absent the targeting nucleic acid, the reporter groups are not retained within the cells.

The targeting nucleic acid can be linked to the reporter group or groups by a variety of methods, including, e.g., covalent bonds, bifunctional spacers (“bridge”) such as, avidin-biotin coupling, Gd-DOPA-dextran coupling, charge coupling, or other linkers.

The reporter groups can be contrast agents such as magnetic particles, e.g., superparamagnetic, ferromagnetic, or paramagnetic particles. Paramagnetic metals (e.g., transition metals such as manganese, iron, chromium, and metals of the lanthanide group such as gadolinium) alter the proton spin relaxation property of the medium around them.

There is considerable latitude in choice of magnetic particle size. For example, the particle size can be between about 1 nm and 2000 nm, e.g., between about 2 nm and 1000 nm (e.g., about 200 or 300 nm), or between about 10 nm and 100 nm, as long as they can still be internalized by the cells. Typically, the magnetic particles are nanoparticles. Preferably, within any particular probe preparation, panicle size is controlled, with variation in particle size being limited, e.g. substantially all of the particles having a similar diameter, e.g., in the range of about 30 nm to about 50 nm. Particle size can be determined by any of several suitable techniques, e.g., gel filtration or electron microscopy. An individual particle can consist of a single metal oxide crystal or a multiplicity of crystals.

There are two types of contrast agents useful for MR imaging: and T1 and T2 agents. The presence of T1 agent, such as manganese and gadolinium, reduces the longitudinal spin-lattice relaxation time (T1) and results in localized signal enhancement in T1 weighted images. On the other hand, the presence of a strong T2 agent, such as iron, will reduce the spin-spin transverse relaxation time (T2) and results in localized signal reduction in T2 weighted images. Optimal MR contrast can be achieved via proper administration of contrast agent dosage, designation of acquisition parameters such as repetition time (TR), echo spacing (TE) and RF pulse flip angles.

Specific examples of useful magnetic nanoparticles include monocrystalline iron oxide nanoparticles (MIONs) as described, e.g., in U.S. Pat. Nos. 5,492,814, 4,554,088, 4,452,773, 4,827,945, and Toselson et al., Bioconj. Chemistry, 10:186-191 (1999), superparamagnetic iron oxide particles (SPIOs), ultra small superparamagnetic iron oxide particles (USPIOs), and cross-linked iron oxide (CLIO) particles (see, e.g., U.S. Pat. No. 5,262,176).

MIONs can consist of a central 3 nm monocrystalline magnetite-like single crystal core to which are attached an average of twelve 10 kD dextran molecules resulting in an overall size of 20 nm (e.g., as described in U.S. Pat. No. 5,492,814 and in Shen et al., “Monocrystalline iron oxide nanocompounds (MION): Physicochemical Properties,” Magnetic Resonance in Medicine, 29:599-604 (1993)), to which nucleic acids can be conjugated for targeted delivery.

The dextran/Fe w/w ratio of a MION can be, e.g., about 1.6:1. R1=12.5 mM sec⁻¹, R2=45.1 mM sec⁻¹ (0.47 T, 38° C.). Relaxivity in an aqueous solution at room temperature and 0.47 Tesla can be: R1 ˜19/mM/sec, R2 ˜41/mM/sec. MIONs elute as a single narrow peak by high performance liquid chromatography with a dispersion index of 1.034; the median MION particle diameter (of about 21 nm as measured by laser light scattering) corresponds in size to a protein with a mass of 775 MD and contains an average of 2064 iron molecules.

The physicochemical and biological properties of the magnetic particles can be improved by crosslinking the dextran coating of magnetic nanoparticles to form CLIOs to increase blood half-life and stability of the reporter complex. The cross-linked dextran coating cages the iron oxide crystal, minimizing opsonization. Furthermore, this technology allows for slightly larger iron cores during initial synthesis, which improves the R2 relaxivity. CLIOs can be synthesized by crosslinking the dextran coating of generic iron oxide particles (e.g., as described in U.S. Pat. No. 4,492,814) with epibromohydrin to yield CLIOs as described an U.S. Pat. No. 5,262,176.

The magnetic particles can have a relaxivity on the order of 35 to 40 in mM/sec, but this characteristic depends upon the sensitivity and the field strength of the MR imaging device. The relaxivities of the different reporter conjugates can be calculated as the slopes of the curves of WIN and 1/T2 vs. iron concentration; T1 and T2 relaxation times are determined under the same field strength, as the results of linear fitting of signal intensities from serial acquisition: (1) inversion-recovery MR scans of incremental inversion time for T1 and (2) SE scans of a fixed TR and incremental TE. Stability of the conjugates can be tested by treating them under different storage conditions (4° C., 21° C., and 37° C. for different periods of time) and performing HPLC analysis of aliquots as well as binding studies.

In some embodiments, the paramagnetic label is a metal chelate. Suitable chelating moieties include macrocyclic chelators such as 1,4,7,10-tetrazazcyclo-dodecane-N,N′,N″,N′″-tetraacetic acid (DOTA). For use in vivo, e.g., as MR contrast agents in a human patient, gadolinium (Gd³⁺), dysprosium (Dy³⁺), and europium are suitable. Manganese can also be used for imaging tissues other than in the brain. In other embodiments, CEST (Chemical Exchange Saturation Transfer) can be used. The CEST method uses endogenous compounds such as primary amines as reporter groups that can be linked to the ODN.

Other suitable reporter groups are labels such as near infrared fluorophores, indocyanine green (ICG). Cy3 5.5, and quantum dots, which can be linked to the targeting nucleic acid and used in optical imaging techniques, such as diffuse optical tomography (DOT) (see, e.g., Ntziachristos et al., Proc. Natl. Acad. Sci. USA, 97:2767-2773, 2000). Other fluorescent labels, such as Fats, Texas Red, and Rhodamine can also be linked to the targeting nucleic acid. Radionuclides, such as ¹¹C, ¹³N, ¹⁵O or can be synthesized into the targeting nucleic acids to form the reporter conjugates. In addition, various known radiopharmaceuticals such as radiolabeled tamoxifen (used, e.g., for breast cancer chemotherapy) and radiolabeled antibodies can be used. For example, they can be coated with dextran for attachment to the targeting nucleic acids as described herein. These radio-conjugates have application in positron emission tomography (PET). Radioisotopes, such as ³²P, ³³P, ³⁵S (short half-life isotopes) (Liu et al. (1994) Ann. Neurol., 36:566-576), radioactive iodine, and barium can also be integrated into or linked to the targeting nucleic acid to form conjugates that can be imaged using X-ray technology.

Note that two or more reporter groups, of the same or different kinds, can be linked to a single targeting nucleic acid.

The targeting nucleic acids are typically double-stranded oligonucleotides of up to 6, 7, 8, 9, 10, 11, 12, 15, 18, 20, 23, 25, 26, 27, or 30 nucleotides in length and designed to bind to the target protein (e.g., if present in sufficient numbers in a cell). They can be protected against degradation, e.g., by including phosphorothioate during synthesis.

The reporter group and the targeting nucleic acid are linked to produce the reporter conjugate using any of several known methods. For example, if the contrast agent is a MION, this molecule can be linked to a nucleic acid by phosphorothioating the oligonucleotide and labeling it with biotin at the 5′ end of one or both strands. The dextran coated MION can be activated and conjugated to the biotin-labeled oligonucleotide using avidin based linkers, such as NeutrAvidin® (Pierce Chem.).

In addition, liposomes, lipofectin, and lipofectamine can be used to help get the entire conjugate into a cell.

Various imaging modalities and corresponding reporter groups are reviewed and described in Min et al., Gene Therapy, 11:115-125 (2004), which is incorporated herein by reference in its entirety, including the references it cites.

### Targeting Nucleic Acids

The targeting nucleic acid can include one or more sequences with at least 80% (e.g., at least 85%, 90%, 95%, 98%, or 99%) sequence homology (identity) with a consensus (predicted, or known) sequence to which the target protein binds. For example, at least 4 contiguous nucleotides (e.g., at least 5, 6, 7, 8, 10, 12, 14, 15, 16, or 20 contiguous nucleotides) in the ODN are at least 80% identical to a consensus, predicted, or known sequence to which the target protein binds. The nucleic acids can additionally include heterologous nucleotides (e.g., at least 1, 2, 3, 4, 5, 6, 7, 8, 10, or 12 nucleotides) 5′ and 3′ of the sequence to which the target protein hinds. Typically, where the nucleic acids are double stranded, these heterologous sequences will include complementary bases of A and T; C and G; A and I (inosine), or substituted bases pairs.

Consensus, predicted, and known sequences that bind specifically to target proteins can be found in the literature or in various databases, such as the TRANSFAC® database (BIOBASE, Beverly, Mass.) (Heinemeyer et al. Nucl. Acids Res., 26:364-370, 1998) and the object-oriented Transcription Factors Database (ooTFD) (www.ifti.org/ootfd) (Ghosh, Nuc. Acids Res., 28:308-310, 2000). Additional information on mutated forms of transcription factors and transcription factors involved in pathological states can be found in the IARC TP53 mutation database (Olivier et al., Hum. Mutat., 19:607-14, 2002) and the Patho® Database (BIOBASE, Beverly, Mass.).

Exemplary nucleic acid sequences that bind specifically to nucleic acid binding proteins include those that bind to Activator Protein-1 (TGACTCA; SEQ ID:NO:1), cyclic AMP responsive elements (TGACGTCA; SEQ ID NO:2), specificity protein-1 (CCCGCC; SEQ ID NO:3), and Nuclear Factor-kappa beta (GGGGACTTTCC; SEQ ID NO:4).

In other embodiments, the targeting nucleic acid can include a specific nucleic acid structure (e.g., a Holliday junction, cruciform, stem loop, lariat, triple helix, nucleosome, methylation, DNA/RNA heterodimer, 3′ or 5′ overhang, a single-stranded nucleic acid, or other structure) to which the target protein binds.

A targeting nucleic acid or a portion thereof, can be isolated using standard molecular biology techniques. Furthermore, targeting nucleic acids can be prepared by standard synthetic techniques, e.g., using an automated DNA synthesizer. A targeting nucleic acid can be constructed using chemical synthesis and enzymatic ligation reactions using procedures known in the art. Targeting nucleic acids can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

### Methods or Administration

For administration, e.g., to an experimental rodent or human patient, a reporter conjugate can be diluted in a physiologically acceptable fluid such as buffered saline, dextrose or mannitol. Typically, the solution is isotonic. Alternatively, the conjugate can be lyophilized and reconstituted with a physiological fluid before injection. The conjugate can be administered parenterally, e.g., by intravenous (IV) injection, subcutaneous injection, or intra-muscular administration, depending on the tissue to be imaged. For imaging the brain, a useful route of administration is the intracerebroventricular (ICV) route. When administered intravenously, the conjugate can be administered at various rates, e.g., as rapid bolus administration or slow infusion.

When administered by IV injection and superparamagnetic iron particles are used as the paramagnetic label, useful dosages are between about 0.1 and 10.0 mg of iron per kg, e.g., between 0.2 and 5 mg/kg for a 1.5 Tesla medical scanner. As is known in this art, there is a field dependence component in determining the contrast dosage. Doses of iron higher than 10 mg/kg should be avoided because of the inability of iron to be excreted. These types of contrast agents can be used at a dosage of 0.001 to 0.1 mg/kg body weight for ICV administration in the rodents.

When administered by IV injection and chelated gadolinium is used as the paramagnetic label, the dose will be between 10 micromoles and 1000 micromoles gadolinium/kg, e.g., between 50 and 100 micromoles gadolinium/kg. Doses above 1000 micromoles/kg produce hyperosmotic solutions for injection.

The new reporter conjugates will shorten the relaxation times of tissues (T1 and/or T2) and produce brightening or darkening (contrast) of MR images of cells, depending on the tissue concentration and the pulse sequence used. In general, with highly T2 weighted pulse sequences and when iron oxides are used, darkening will result. With T1 weighted pulse sequences and when gadolinium chelates are used, brightening will result. Contrast enhancement will result from the selective uptake of the conjugate in cells that contain the target gene.

If delivered systemically, paramagnetic metal chelate-type conjugates will show renal elimination with uptake by the liver and spleen, and to a less degree by other tissues. Superparamagnetic iron oxide crystal-type conjugates are too large for elimination by glomerular filtration. Thus, most of the administered conjugates will be removed from the blood by the liver and spleen. Superparamagnetic iron oxides are biodegradable, so the iron eventually will be incorporated into normal body iron stores.

Various reporter groups for medical imaging are routinely administered to patients intravenously, but can also be delivered by intra-peritoneal, intravenous, or intra-arterial injection. All of these methods can deliver the new reporter conjugates throughout the body except to the brain due to the existence of the blood brain barrier (BBB). To bypass the BBB, one can use one of several methods known to the skilled in the art, e.g., ICV, intrathecal injection into the Cisterna Magna, or intra-arterial injection into the ascending aorta, followed by the transient breakage of BBB (e.g., via mannitol infusion). In certain situations, the BBB may be already breached because of a specific disorder, such as certain cancers.

### Methods of Imagine

MR imaging can be performed in live animals or humans using standard MR imaging equipment, e.g., clinical, wide bore, or research oriented small-bore MR imaging equipment, of various field strengths. Imaging protocols typically consist of T1, T2, and T2* weighted image acquisition. T1 weighted spin echo (SE 300/12), T2 weighted SE (SE 5000/variable TE) and gradient echo (GE 500/variable TE or 500/constant TE/variable flip angles) sequences of a chosen slice orientation at different time points before and after administration of the reporter conjugate.

To determine the in vivo distribution of a particular reporter conjugate, biodistribution studies and nuclear imaging can be carried out using excised tumors of animals that have received a single dose of labeled reporter complex. e.g., MION-s-ODN. The same assay can be used to analyze the biodistribution of other new reporter conjugates.

To determine whether expression of a specific target protein. e.g., a therapeutic transgenic protein, can be detected with a particular reporter conjugate, animals receive an infusion of the conjugate. After injection, differences in R2* maps (inverse of T2* maps) are determined after a pre-defined period of time. If significant, the reporter conjugate can be used in clinical imaging of that specific transgene. Biodistribution studies can be used to show a higher concentration of the reporter conjugate in cells expressing the target protein compared to matched cells that do not express (or over-express) the target protein in the same animal.

This image evaluation technique can also applied to other imaging modalities such as PET, X-ray, and DOT, in which radionuclides, radioisotopes, and/or fluorescent probes are detected. Such other imaging modalities, and their corresponding reporter groups, are described in Min et al. (Gene Therapy, 11:115-125 (2004)).

### Applications

Examples of known nucleic acid binding proteins that can be targeted using the methods and compositions described herein include Activator Protein-1 (AP-1) (a heterodimer of Fos and Jun proteins), Activator Protein-2 (AP-2), cyclic AMP responsive element proteins (CREP), specificity proteins (e.g., SP-1, SP-2, SP-3), nuclear factor kappa beta protein (NF-κB, Ras, p53, E2F transcription factors (e.g., E2F-1, E2F-2, E2F-3, E2F-4, E2F-5), Forkhead transcription factors (e.g., FOXO1, FOXO1a, FOXO3a, FOXC1, FOXC2, FOXP2), Kruppel like factors (e.g., KLF4, KLF5), interferon regulatory factors (e.g., IRF-1. IRF-2, IRF-3), retinoid X receptors (e.g., retinoid X receptor gamma), signal transducer and activator of transcription proteins (e.g., STAT1, STAT3, STAT5), DATA transcription factors (e.g., GATA-1, DATA-2, GATA-3, GATA-4. GATA-5. GALA-6). Polycomb silencers, members of the Zif268/NGF-I family (e.g., Zif268 (Egr1). Egr2, Egr3, NGF-IC, WT1), DMP1, Spi-B, Evi1, Hypoxia-inducible factors (e.g., HIF-1), proteins of the ets family (e.g., ETS, ERR ELK-1, SAP-1, EHF, MEF), VHL, Twist, BRCA1., PEA3, Myc, CtIP, ER, ZBRK1, Goosecoid, Slug, Oct4, Nanog, Stella, Microphthalmia transcription factor (Mitf), Major Cdk9-interacting elongation factor (MCEF), NPAS3, Regulatory factor X4 variant 3 (RFX4_v3), POU domain, class 3, transcription factor 3 (Pou3f3), Pitx3, CCAAT/enhancer binding protein β (C/EBPβ), E2F1. TReP-132, retinoid-related orphan nuclear receptor alpha(RORa), upstream stimulatory factor (USF). Elk-1, Gli-1, Nurr-1, Fe65, YY1, LBP-1c/CP2/LSF (LBP-1c), -FosB, cyclic-AMP response-element-binding protein (CREB), Nac-1, glucocorticoid receptors cancer-associated transcription factors, brain-associated transcription factors, and nuclear receptors. A brain associated transcription factor can be one found in the Functional Genomic Atlas of the Mouse Brain (mahoney.chip.org/Mahoney) or a human homolog thereof.

The new methods and compositions have numerous practical applications. For example, they can be used for imaging nucleic acid binding protein expression in deep organs using MR imaging, and for imaging tumors that over-express certain target nucleic acid binding proteins compared to normal cells. For example, the new methods and compositions can be used to detect expression, overexpression, or activity of oncoproteins or proto-oncoproteins in live animals. The new reporter conjugates can be used to detect expression of an oncoprotein, e.g., a mutant proto-oncoprotein or a mutant tumor suppressor protein, in a tumor or cancerous cell at a very early stage in tumor development. Several oncoproteins (ray, N-myc, C-myc, L-myc, bcl-2, IRF-2) and tumor suppressors (p53, WT1, PEA3, MEF, KLF5, DMP1, FOXO1a, BRCA1, IRF-1), are known in the art.

In other embodiments, the new methods and compositions can be used to detect nucleic acid binding proteins (e.g., p53, NF-κB, AP-1, IRF-3) involved in the process of cell death (e.g., neuronal cell death). MR can be used in real time to visualize expression or activity of nucleic acid binding proteins involved in cell death, e.g., after stroke or associated with other neurological disorders such as Alzheimer's disease or Parkinson's disease.

The new methods and compositions can also be used to detect and/or image nucleic acid proteins involved in learning, memory, and/or addiction (e.g., NPAS3, FOXP2, -FosB, CREB, Egr1, Egr2, Egr3, Nac-1, glucocorticoid receptors, NF-κB). MR images can be taken in real time to detect the expression or activity of nucleic acid proteins involved in learning or memory. e.g., during functional MR imaging on individuals performing learning and memory tasks.

The new methods can also used to image endogenous nucleic acid binding protein expression during development and/or pathogenesis of disease. Additionally, the expression or activity of a specific nucleic acid binding protein (e.g., an activator or repressor protein) within an animal can be directly visualized. Moreover, imaging of nucleic acid binding protein expression by high-resolution MR imaging will have a major impact in the treatment of CNS disease such as brain tumors or neurodegenerative diseases such as Alzheimer's. The new reporter conjugates can be used for in vivo monitoring of nucleic acid binding protein expression or activity associated with such disease states.

The use of reporter conjugates to image cellular nucleic acid binding proteins, e.g., to image protein expression or activity, enables the monitoring of gene therapy where exogenous protein-expressing genes are introduced to ameliorate a genetic defect or to add an additional protein function to cells.

In other embodiments, the new reporter conjugates can be used more generally for non-invasive detection of nucleic acid binding protein expression, cell mapping, gene targeting, phenotyping, and detection of multiple proteins using two or more unique ODNs linked to different unique reporter groups. The new conjugates can also be used to deliver chimeric reporter groups, e.g., two or more different reporter groups linked to the same targeting nucleic acid, to specific cells, with or without the use of antibodies that specifically bind to cell-surface antigens.

In other embodiments, the new reporter conjugates can be used to detect the protein expression of stern cells. Oct4, Nanog, and Stella are transcription factors typically expressed in pluripotent stem cells. Specific patterns of gene and protein expression can arise in differentiating stem cells, depending on the type of stem cell. Stem cells can be visualized, e.g. following implantation (e.g., before, during, or after stern cell therapy) in a subject.

In other embodiments, the new reporter conjugates can be used to detect, visualize, or localize the expression of a transgenic protein in a subject. The expression of a transgenic protein that is expressed conditionally (e.g., from a conditional promoter) or tissue specifically (e.g., from a tissue-specific promoter) can be imaged using the new reporter conjugates.

The new methods can also be used for treatment of a disorder or injury in a patient mediated by a nucleic acid binding protein. e.g., a nucleic acid binding protein described herein. In one non-limiting example, a reporter conjugate described herein can be administered at a sufficient concentration to prevent binding of its target nucleic acid binding protein to its endogenous intracellular target (e.g., chromosomal DNA). For example, reporter conjugates can be used to decrease gene expression mediated by an oncogene (e.g., ras, N-myc, C-myc, L-myc, bcl-2, IRF-2) to treat a cancer. In another example, a reporter conjugate can be used to decrease gene expression mediated by a nucleic acid protein involved in cell death (e.g., p53, NF-κB, AP-1, IRF-3) to reduce cell death, e.g., after stroke or associated with other neurological disorders such as Alzheimer's disease or Parkinson's disease.

### EXAMPLES

The invention is further described in the following examples, which do not limit the scope of the invention described in the claims.

### Example 1

### Preparation of MION-s-ODN

Phosphorothioated ODN (s-ODN) labeled with biotin on the 3′ end were used as the targeting nucleic acid portion of the reporter conjugate. The following s-ODN were made as single-stranded nucleic acids, then hybridized to double-stranded DNA (25 nmol each in 50 at room temperature 10 minutes and stored at −20° C.). Capital letters indicate sequences that hind specifically to the indicated nucleic acid binding proteins.

Activator Protein-1

| | |
|---|---|
| AP-1 | |
| 5′-tccggcTGACTCAtcaagc2-3′-biotin | (SEQ ID NO: 5) |
| biotin-- | |
| 3′-aggccgACTGAGTagttcgc-5′ | (SEQ ID NO: 6) |

Cyclic AMP Responsive Elements

| | |
|---|---|
| CREB | |
| 5′-ctctcTGACGTCAggcaat-3′-biotin | (SEQ ID NO: 7) |
| biotin-- | |
| 3′-gagagACTGCTGTccgtta-5′ | (SEQ ID NO: 8) |

Specificity Protein-1

| | |
|---|---|
| SP-1 | |
| 5′-ctcgcCCCGCCccgatcgaa-3′-biotin | (SEQ ID NO: 9) |
| biotin-- | |
| 3′-gagcgGGGCGGggctagctt-5′ | (SEQ ID NO: 10) |

Nuclear Factor-Kappa Beta

| | |
|---|---|
| NF-κB | |
| 5′-gttgaGGGGACTTTCCcagg-3′-biotin | (SEQ ID NO: 11) |
| biotin-- | |
| 3′-caactCCCCTGAAAGGgtcc-5′ | (SEQ ID NO: 12) |

The reporter group was a dextran-coated contrast agent, a monocrystalline iron oxide nanoparticle (MION), a ultra-small superparamagnetic iron oxide particle (USPIO), or a superparamagnetic iron oxide nanoparticle (SPION) that was activated and conjugated using NeutrAvidin® (Pierce Biotechnology, Rockford, Ill.). Neutravidin-dextran-coated MION particles were covalently hound to the s-ODN to form the novel reporter conjugates.

Functional groups were attached to MIONs, USPIOs, or SPIONs (5 ml at 2 mg iron per ml) in the presence of 10 ml of sodium hydroxide at 3 N, mixed, and 3.48 g of chloroethylamine (final concentration of NaOH is 1.5N, chloroethylamine is 2 M in 15 ml) is added. The mixture was incubated with slow stirring at room temperature overnight in a well ventilated room. The solution was made neutral using HCl or NaOH, followed by filtration and three washings in 20 ml of 100 mM phosphate buffered saline (PBS, pH 7.4) using a membrane with cutoff at 100,000 Dalton (Millipore) to a final volume of 5 ml.

NeutrAvidin® was attached to functional groups on the dextran coating on the MIONs, USPIOs, and SPIONs using an aldehyde-activated dextran coupling kit (Pierce Biotechnology, Rockford, Ill.). Briefly, 20 mg of activated MION, USPIO, or SPION (5 mg/ml) was added to 10 mg NeutrAvidin® (2.5 mg/ml PBS) and the volume adjusted using a phosphate buffered saline (pH 7.4) to a final volume of 10 ml. Then, 0.9 ml of cyanoborohydride (64 mg/ml PBS) was added and incubated overnight at room temperature, followed by three washings in sodium citrate (25 mM, pH 8) using repeat filtrations in filter-membrane (100 kD cut off). The final volume was 5 ml (iron was 3-4 mg/ml). The solution was stable stored at 4° C. in an amber coated and rubber-sealed bottle. The resulting SPION NeutrAvidin® should optimally have one biotin binding site available to biotinylated s-ODN so that a reporter conjugate had a loading capacity of one, that is, one SPION per each s-ODN

Ten microliter of biotinylated phosphorothioated ODN (s-ODN at 1 μmole/ml) was added to 50 of Neutravidin-MION, and the mixture was incubated for at least 30 minutes at room temperature, followed by filtration and washing in filter-membrane (100 kD cut off) to form the complete reporter conjugate MION-s-ODN.

### Example 2

### Delivery of MION-s-ODN Conjugates

Two groups of mice were used in this study, control animals with MION only and mice with the novel conjugate, MION-s-ODN. Anesthesia was induced with ketamine (100 mg/kg, i.p.) plus xylazine (16 mg/kg, i.p.) to male C57bB6 mice (23-25 g, Taconic Farm, N.Y.), and surgery was performed as described previously (Cui et al., 1999), except MION or MION-s-ODN was delivered to the brain via intracerebroventricular route (LR: −1.0, AP: −0.2, DV: −3.0 to the Bregma). Immediately before use, biotinylated s-ODN was conjugated to Neutravidin®-dextran-MION for 30 minutes at room temperature. A total of no more than 2 microliters of artificial cerebrospinal fluid (aCSF) containing either MION-s-ODN or MION-dextran (control) was infused over 5 minutes into the left lateral ventricle guided by a stereotaxic device. At fixed times after delivery (30 minutes, 3 hours for control, and additional 24 and 48 hours for animals that receive MION-s-ODN); the animals were anesthetized, except the 30-minute time point, with pure O₂ plus 2% halothane (800 ml/min flow rate) and placed in a home-built cradle for MR scanning.

### Example 3

### MRI of Mouse Brain After the Delivery of MION-s-ODN

All scanning was done in a 9.4 T MRI system (Bruker-Avance). A home built 1 cm transmit/receive surface coil was placed on the head of the animal. The MRI scanning protocol at each time point was as follows: serial multi-slice 12 weighted gradient echo (GE) (TR=500 ms, TE=2.3, 3, 4 and 6 ms, flip angle 30, 128×128 pixels, 0.5 mm slice, 20 slices, 15 mm FOV, 4 averages) were performed along the axial and sagittal planes. Image analysis is performed using MRVision® software (MRVision Co, Winchester, Mass.), MATLAB® (The MathWorks Inc., Natick, Mass.), and in-house software to construct T2* maps. In general, these acquisition sequences were readily available in any clinical MRI system. T2* maps can be calculated by the data processing software package included in the imaging system.

Regions of interest (ROI) were extracted, in particular along the cortices of the brain, close to as well as away from the ventricle and the injection sites. T2* maps (or its reciprocal map, R2*) were obtained at pre-determined time points (such as less than 30 minutes after infusion, and either at 3 hours after infusion (to look for wash out) or one clay after infusion (to look for retention)).

### Example 4

### MRI of Mouse Brain After the Delivery of Non ODN-Conjugated MION

To determine the specificity of s-ODN, a control conjugate was produced with either biotinylated dATP, dUTP, or a scrambled nucleic acid sequence in place of the target nucleic acid sequence. MION conjugated to the control nucleic acids were infused into mice as described above. Washout of the MION was observed within three hours. Therefore. MION can be retained in the brain and the retention is dependent on ODN labeling.

### Example 5

### Quantification of MION-s-ODN Uptake and Gene Expression in Mouse Brain

T2* values collected from each animal were compared between two time points within similar regions of the brain: less than 30 minutes after the infusion procedure and more than 24 hours after infusion. ANOVA statistical analysis was performed in the Prism Graph Pad software packages.

R2* (1/T2*) values were compared in contralateral cortical regions from selected brain slices of mice injected with MION-sODN and MION-dextran immediately (<30 minutes) or 1 day after infusion. Due to the small size of a mouse brain and the interference image artifact (e.g., extensive region of great signal reduction) caused by the air-tissue interface in ears and trachea as well as intraventricular retention of WON, selection of the brain slices and regions of interest was limited to areas of least artifact.

Immediately following infusion, there was no significant MION-retention (relaxivity in second-1) (p>0.05) in the contralateral cortices in animals that received MION-dextran and MION-s-ODN, suggesting equal delivery of MION. One day after infusion, MION-retention in the contralateral cortex to the infusion site (within one mm) was significantly higher (two-way ANOVA, p<0.01) in the animals that receive MION-s-ODN than in those that received MION-dextran only. MION retention in animals that receive MION conjugated to control nucleic acids was not significantly different from those that received MION-dextran.

### Example 6

### Postmortem Tissue Preparation

At various given times before or after MION-sODN or MION-dextran infusion, the animals were anesthetized for transcardial perfusion with 20 ml heparinized saline (2 units) at the rate of 10 ml/min, followed by 20 ml of 4% paraformaldehyde (PFA) in 0.1 M phosphate buffer (PBS), pH 7.4 at a rate of 10 ml/min. The brain was removed and kept in the same perfusate for at least 4 hours at 4° C., followed by chase and storage in PBS with 20% sucrose solution. The brain was then processed, and embedded in paraffin. Coronal tissue slices (each at 6-microns) were cut posterior or anterior to the injection site for immunohistochemical staining. Paraffin embedded tissue sections were de-waxed using xylene, chloroform, and dehydration in serial ethanol (100%, 95%, and then 75%).

### Example 7

### Detection of Intracellular Presence of MION

The presence of iron oxide was detected using Prussian blue, followed by fast nuclear red counter staining (Fisher Chem. Co).

The presence of iron oxide (blue-green color using Prussian blue staining for iron) and nuclear fast red for nuclei counter-stain (pink-red) was observed the brains of animals that receive MION-s-ODN. No iron oxide was observed in animals that receive only MION-dextran.

### Example 8

### Elevation of AP-1 Activity in Stroke Model

Neural ischemia was induced in mice by transient bilateral carotid occlusion (BCAO). All procedures and animal care practices adhered strictly to Association for Assessment and Accreditation of Laboratory Animal Care (AAALAC), Society for Neuroscience, and institutional guidelines for experimental animal health, safety, and comfort. After anesthetizing male C57Black6 mice (25±2 g, Taconic Farm, Germantown, N.Y.) with a mixture of ketamine (80 mg/kg, i.p.) and xylazine (12 mg/kg, i.p.), a midline ventral incision was made in the neck. Both common carotid arteries were isolated, freed of nerve fibers, and occluded for 30 minutes using nontraumatic aneurysm clips (Fine Science Tools, Inc). The occlusion was released for reperfusion as described previously (Liu et al., J. Neurosci., 16:6795-6806, 1996). Sham-operated animals underwent the same surgical procedure except for actual execution of artery occlusion. Body temperature was monitored and maintained at 37±1° C. throughout surgery and the immediate postoperative period, until the animals recovered fully from anesthesia.

Transcripts of cFos and actin were detected in the ischemic and sham-operated mice with antisense nucleic acids against cfos (5′-catcatggtcgtggtttgggcaaacc-3′: SEQ ID NO:13; Cui et al., J Neurosci., 19:1335-44, 1999) and actin. (5′-gagggagagcatagccct-cgtagatg-3′: SEQ ID NO:14; Alonso et al., J. Mol. Evol., 23:11-22, 1986)). AP-1 nucleic acid binding protein activity was detected using the double stranded nucleic acid 5% tccggcTGACTCAtcaagcg-3′ (SEQ ID NO:5). The oligo nucleotides conjugated to superparamagnetic iron oxide nanoparticles (SPION) were delivered to the brains of the mice via intracerebroventricular route (LR: −1.0, AP: −0.2, DV: −3.0 to the Bregma).

In vivo image acquisition was performed with 9.4 Tesla MRI (Bruker Avarice system. Bruker Biospin MRI. Inc., Billerica, Mass.) at different post-infusion time points. Animals were anesthetized with 2% halothane in pure O₂ (800 ml/min flow rate), and blood oxygenation levels were monitored by pulse oximetry. A one-inch surface coil was used for excitation and signal detection. Serial gradient echo (GE) images were used with a constant repetition time (TR=500 ms) and incremental echo spacing (TE=3, 4, 6 ms) to construct R2* maps (R2*=1/T2*) for a 500-μm thick slice at a resolution of 120 μm in the image plane.

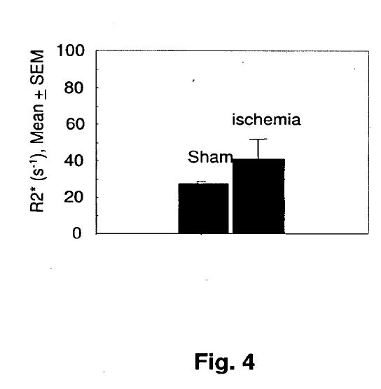

The MR images were co-registered, and the mean R2* maps of sham-operated and BCAO-treated animals were computed using in-house software (Martinos Center for Biomedical Imaging at MGH). Corresponding brain slices were subtracted (cerebral ischemia minus sham-operated), and the percent decrease in R2* was computed. FIGS. 3A and 3B show R2* maps of sham-operated and ischemic animals infused with oligonucleotides for detecting c-Fos and actin transcripts, respectively. R2* subtraction maps are shown in FIGS. 3C and 3D. FIG. 3E depicts R2* subtraction maps of AP-1 nucleic acid protein binding activity in two ischemic animals. FIG. 4 shows R2* values representing AP-1 nucleic acid binding protein in the somatosensory cortices in sham-operated (Sham) and cerebral ischemia (Ischemia) in live animals (n=2). This example demonstrates that nucleic acid binding protein activity can be detected in live animals using reporter conjugated nucleic acids.

### Example 9

### Elevation of AP-1 Activity in Mice Receiving Amphetamine

It was known that exposure to amphetamine (Amph) induces behavior sensitization through structural plasticity, triggering long-lasting neuronal adaptations that lead to compulsive addictive behavior in humans and in animal models of sensitization. Giving Amph to rodents has been used to induce symptoms mimics bipolar disorder and psychosis in humans. The abnormal behavior in rodents includes stereotypical sniffing, rearing, and increased locomotion. While the causal relationship of hyperlocomotion and altered gene activities after Amph stimulation is not fully understood, the induction of protein products of the immediate early genes, e.g., c-fos, FosB, and delta FosB (ΔFosB) are localized within the dopaminergic pathway of the brain, including the medial prefrontal cortex (mPFC), nucleus accumbens (NAc) and caudate putamen (CPu). The protein product of immediate early gene forms a duplex protein of Fos-Jun families. This duplex protein binds to promoter regions of several genes having a consensus sequence for the duplex protein. Upon binding, the duplex protein activates the transcription of a corresponding gene. Therefore, this duplex protein is called activator protein-1 or AP-1. Conventional methods for detecting AP-1 activities are limited to using in vitro cell extract and gel electrophoresis.

We developed brain probe that detects AP-1 protein in vivo by using double stranded oligodeoxynucleotide (ds-ODN) with AP-1 consensus sequence linking to SPION for MRI (see Example 1 above).

Briefly, seven mice were anesthetized by pure O₂-2% halothane at a flow rate of (800 ml/min) and injected with MRI contrast probes (SPION-AP1ds, SPION-fosB or SPION-Ran, at 84 pmol iron per kg) via an ICV delivery route. Each mouse was administered Amph (4 mg/kg, n=3) or saline (vehicle, 4 ml/kg, n=4) given (i.p.) three hours later. Three hours later, hyperlocomotion assay was assessed. MRI was conducted at four hours after administration of Amph or saline.

For in vivo MRI acquisitions, all mice were anesthetized and their ears filled with toothpaste to minimize susceptibility artifact caused by the tissue-air interface. All in vivo MRI acquisitions were performed with a 9.4 Tesla MRI scanner (Bruker-Avance system) in the manner described above. The MRI scanning protocols at each time point included multi-slice gradient echo (GE) imaging sequences (TR=500 ms, TE=3, 4, 6, 8 and 10 ms, flip angle=30, 128×128 pixels, 0.5 mm slice, 20 slices, 15 mm FOV, 2 averages) along the axial and sagittal planes. For voxel-wise and ROI comparison, images were automatically and manually aliened using nine degrees of freedoms (3 each): rotations, translations, and inflations. Fine-tuning of alignment was performed by visual comparison to the template images, focusing on obvious structures such as the corpus callosum and outlines of the ventricles. R2* maps were constructed from the aligned images (with incremental TEs). R₂* (inverse of T₂*) maps were calculated using pixel-wise linear fitting from the set of images with same TR and incremental TEs based on equation M=M₀×exp (−TE/T₂*). ROIs were outlined and mean R2* values extracted according using MRVision (MRVision Co, Winchester. MA). Mean R₂* values and standard errors from different animals groups were obtained and analyzed statistically. The mean and standard error of the mean (SEM) were computed from the averaged values in each group of animals, and compared the statistical significance of these values using test (one tail, type II or equal variant, GraphPad Prism IV, GraphPad Software, Inc, San Diego. CA). A p value of less than 0.05 was deemed to be statistically significant. Following MRI scanning, postmortem brain samples were excised for immunohistochemical analysis of probe uptake or antigen staining.

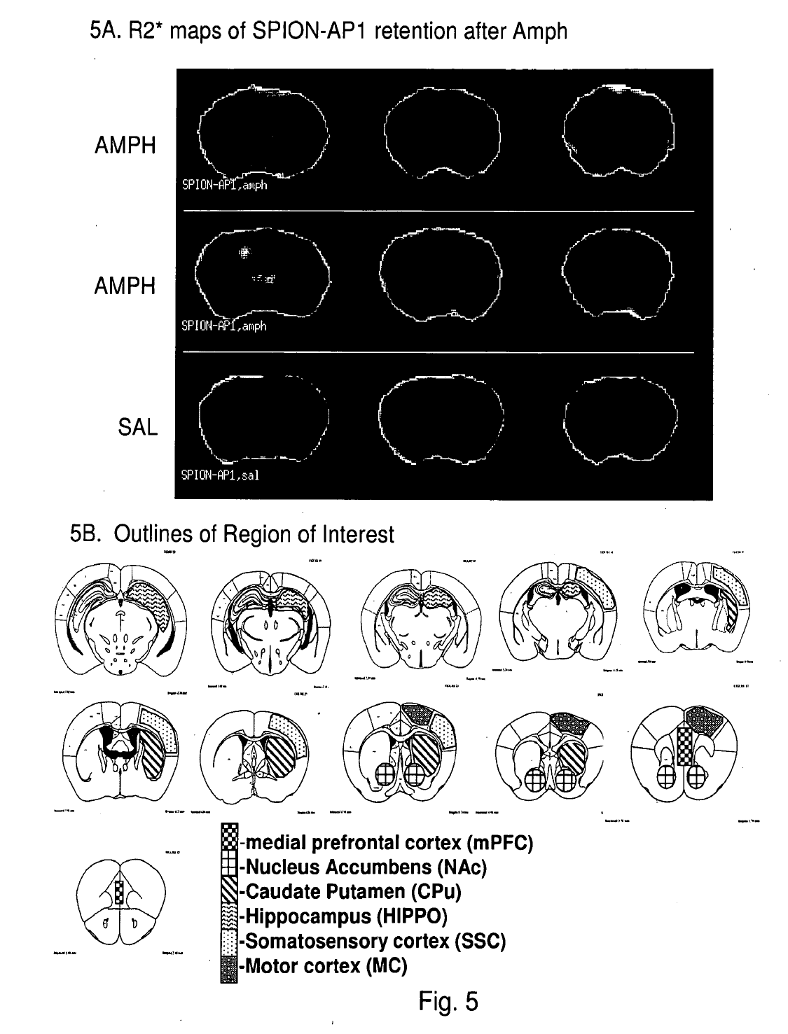

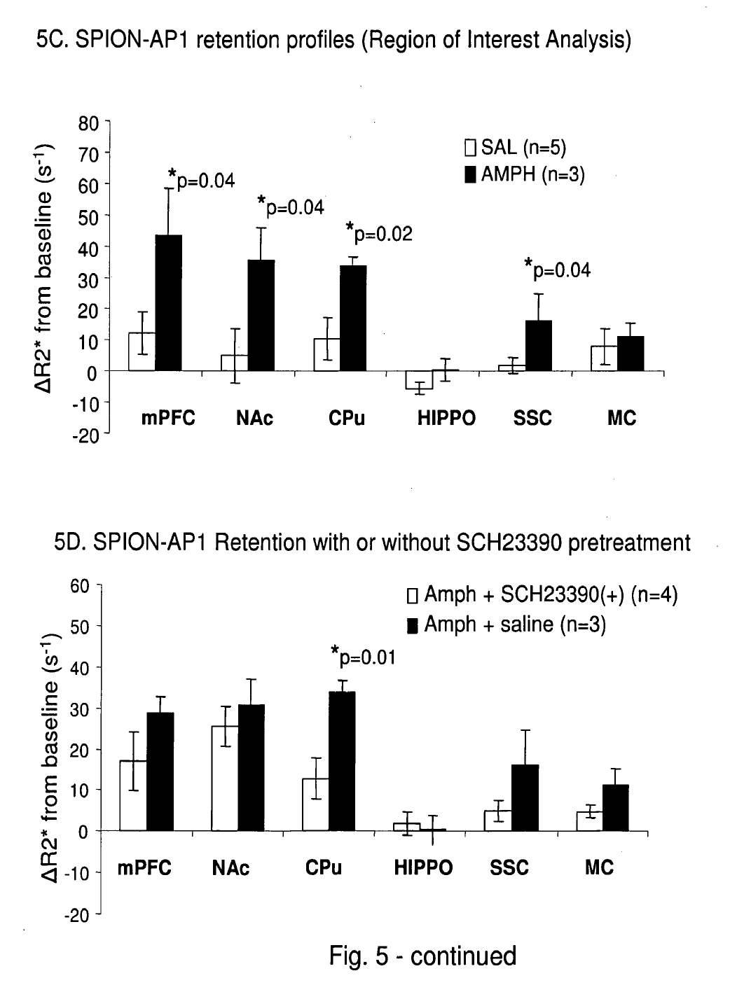

We computed R2* maps, and analyzed mean R₂* values in selected regions of interest (ROIs, located from −3.16 mm to 1.7 mm referenced to the bregma) of the brain in these live animals. Typical R2* maps of SPION-AP1ds with or without Amph stimulation are shown in FIG. 5A (corresponding to square enclosed brain atlas templates shown in FIG. 5B). We observed localized signal enhancement in the AMPH brains (AMPH), indicative of elevated SPION probe retention compared to the control brain (SAL).

To investigate regionally specific signal elevation related to Amph stimulation, we perform statistical analysis in mean. R2* values of selected brain regions. Outlines of regions of interest (ROIs) used for statistical analyses superimposed on a mouse brain atlas (Paxinos et al. 2001. The Mouse Brain in Stereotaxic Coordinates, Academic Press Limited, London) are shown in FIG. 5B. To reduce possible bias in the analysis, we analyzed most of the ROIs in the contralateral hemisphere to the infusion hemisphere except for the mPFC and NAc. As shown in FIG. 5C. AP-1 activity was significantly elevated in a regionally specific manner in key regions of the dopaminergic pathway (mPFC. NAc and CPu) and SSC, but not in the hippocampus (HIPPO) and motor cortex (MC). Regions of elevated SPION-AP1ds retention due to Amph stimulation overlapped regions of enhanced SPION-cfos retentions, except for the somatosensory cortex (SSC).

We then tested whether SPION-AP1ds signal enhancement was indeed due to Amph stimulation. In this study, we pretreated two groups of mice respectively with subcutaneous injection of SCH23990 (an D1/D5 receptor antagonist, 0.1 mg/kg, n=4, SCH23390/AMPH) and with saline only (n=3, SAL/AMPH), 40 min prior to Amph stimulation, which occurred three hours after ICV infusion of SPION-AP1ds probe.

MRI was performed four hours after Amph stimulation. We observed regions of reduced SPION-AP1ds retention in brain regions depicted as hotspots on the subtraction maps between mean R2* maps of SAL/AMPH and SCH23390/AMPH. The Regions of Interest Analyses (FIG. 5D) showed that only the CPu region had a significant reduction.

This result suggests that the effect of SCH23990 upon AP-1 is more localized to the nigrostriatal neurons at the CPu, although SCH23390 antagonizes D1/D5 receptors distributed throughout the neurons of the dopaminergic pathway.

### Example 10

### MRI Detecting of Amph-Induced fosB mRNA

Since AP-1 proteins are heterodimers of FOS and JUN protein families, in this example, study was conducted to co-localize AP-1 activity profile to FosB expression based on its in RNA expression profiles after Amph stimulation.

An sODN-fosB probe was designed to target fosB mRNA. This probe, SPION-fosB (5′-CCTTAG CGGATGTTGACCCTGG-3% SEQ ID NO: 15), is complementary to the sequence from NT 1925 to 1.946 of mmFosB (Accession No. X14897). The phosphate backbones were modified by phosphorothioate. Using this probe, a fragment of 146 base pairs was amplified from fosB cDNA, but not from the closely related ΔfosB cDNA., demonstrating the probe's discrimination between cDNA of fosB and ΔfosB. This sODN-fosB probe thus provided the specificity to identify endogenous fosB mRNA.

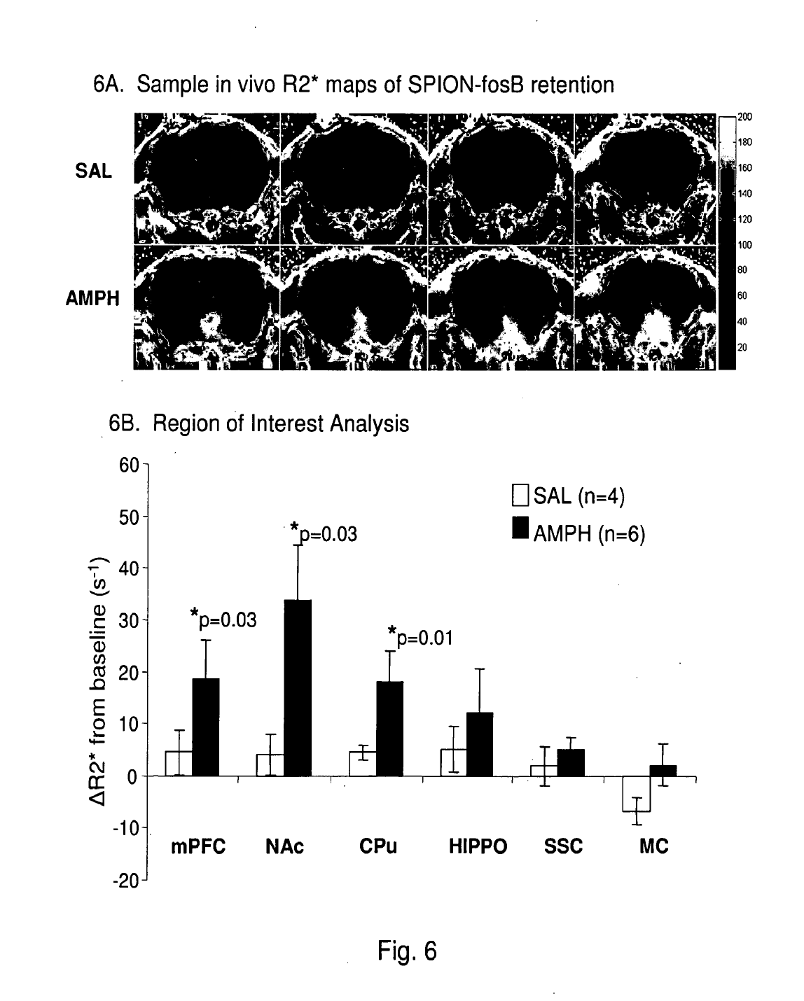

ICV probe infusion, Amph stimulation, and MRI acquisition were conducted in the same manner descried above. We compared the retention profiles of SPION-fosB in AMPH (n=6) and SAL groups (n=4) based on R2* maps computed from MRI in live mice. FIG. 6A shows representative R₂* maps acquired after Amph injection, compared to those of SAL injection. R2* maps of Amph stimulated brains showed enhanced SPION-fosB signals in a regionally specific manner, which was validated by ROI analysis (FIG. 6B). Amph stimulation resulted in significant signal elevations of SPION-fosB in mPFC, NAc and CPu, similar to what was observed in the case of SPION-AP1ds, but not in HIPPO. SSC and MC.

Amph-induced fosB mRNA increase was then confirmed by histology analysis. The analysis was conducted using FITC-sODN targeting via in vivo hybridization and ex vivo imaging using fluorescence microscopy according to methods described in Liu, et al. 2007, J Neurosci 27, 713-722. Consistent with observation from in vivo MRI assessment, we observed an elevated retention in tissue samples from AMPH group, compared to those from SAL group, with the majority of the FITC signal in the cytoplasm. On the other hand, infusion of the control probe with no intracellular target, sODN-Ran-FITC resulted in no enhanced retention profiles after AMPH stimulation.

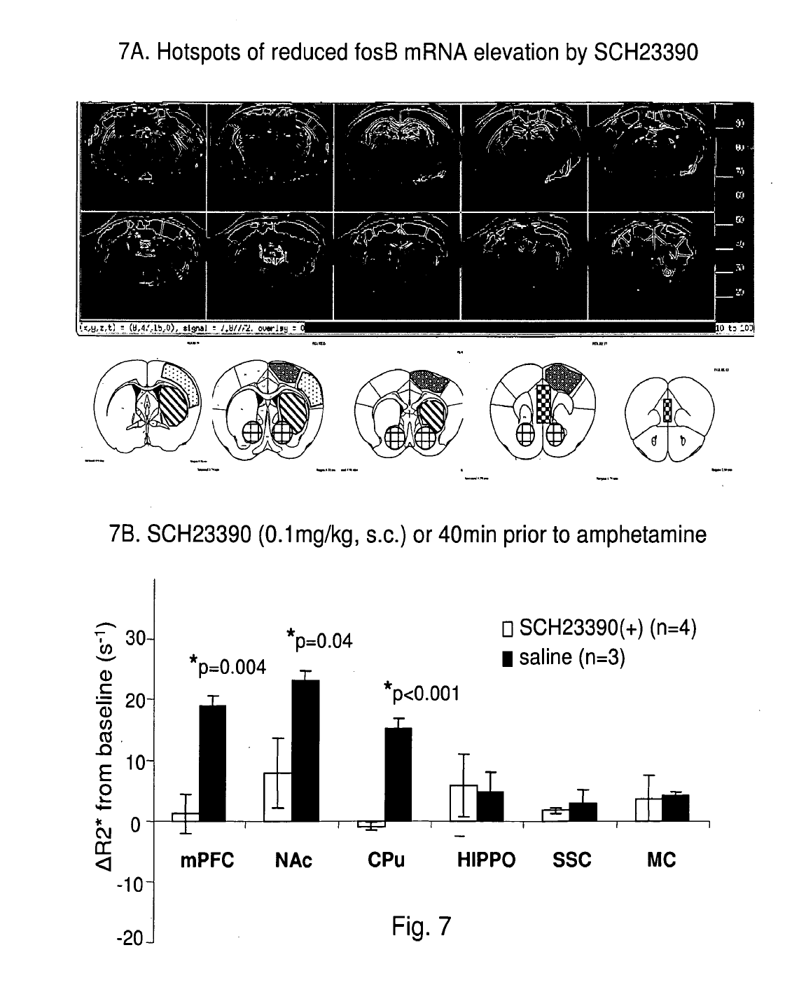

We further investigated whether or not SPION-fosB signal enhancement was indeed due to Amph stimulation. We employed similar antagonist pretreatment paradigm described above and compared the R2* maps between SCH23390/AMPH (n=4) and SAL/AMPH (n=3) groups. We observed regions of reduced SPION-fosB retention in several brain regions depicted as hotspots on the subtraction maps between mean R2* maps of SAL/AMPH and SCH23390/AMPH in percent decrease (FIG. 7A, peudocoloar bar to the right, ranging from −10 and −100%) which were localized to the general areas of the CPU and mPFC. The region of interest analysis showed that SCH23390 reduced AMPH-induced fosB mRNA expression in the mPFC, NAc and CPu with a greatest reduction in the CPu (FIG. 7B).

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features. From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

1. A method of detecting a nucleic acid binding protein in a tissue in vivo, the method comprising
obtaining a reporter conjugate comprising a targeting nucleic acid linked to a reporter group, wherein the targeting nucleic acid binds specifically to a nucleic acid binding protein to be imaged;
administering the reporter conjugate to the tissue in an amount sufficient to provide a detectable image;
allowing sufficient time to pass to allow a sufficient amount of unbound reporter conjugate to leave the tissue; and
imaging the tissue, wherein a detectable image of the reporter group in the tissue indicates the presence of the nucleic acid binding protein.

2. The method of claim 1, wherein the nucleic acid binding protein comprises an activator or repressor protein, and the targeting nucleic acid comprises a double-stranded consensus binding sequence of the protein.

3. The method of claim 1, wherein the nucleic acid binding protein is a therapeutic protein previously delivered to the tissue.

4. The method of claim 1, wherein the tissue is brain tissue.

5. The method of claim 1, wherein the tissue is heart, lung, liver, pancreas, spinal cord, prostate, breast, gastrointestinal system, ovary, or kidney tissue.

6. The method of claim 1, wherein the reporter group is a superparamagnetic iron oxide particle with a maximum diameter between 1 nm and 2000 nm.

7. The method of claim 1, wherein the tissue is in a human patient.

8. The method of claim 1, wherein the reporter conjugate is administered by intravenous injection.

9. The method of claim 1, wherein the reporter conjugate is administered via intra-cerebroventricular infusion.

10. A reporter conjugate for detecting a cellular nucleic acid binding protein comprising a single double-stranded targeting nucleic acid that binds specifically to a nucleic acid binding protein, linked to one or more superparamagnetic iron oxide particles with a maximum diameter between 1 nm and 1000 nm.

11. The reporter conjugate of claim 10, wherein the particle is a monocrystalline iron oxide nanoparticle (MION), ultra small superparamagnetic iron oxide particle (USPIO), superparamagnetic iron oxide nanoparticle (SPION), or cross-linked iron oxide (CLIO) particle.

12. The reporter conjugate of claim 10, wherein the maximum diameter of the particle is between 10 nm and 100 nm.

13. The reporter conjugate of claim 10, further comprising cross-linked dextran linked to the particle.

14. A method of treating a disorder in a patient, the method comprising:
obtaining a conjugate comprising a targeting nucleic acid linked to a therapeutic agent, wherein the targeting nucleic acid binds specifically to a target nucleic acid binding protein corresponding to a target organ or tissue; and
administering the conjugate to a patient in an amount sufficient to treat the disorder.

15. The method of claim 14, wherein the disorder is a cancer.

16. The method of claim 15, wherein the targeting nucleic acid preferentially binds to an oncoprotein.

17. The method of claim 15, wherein the targeting nucleic acid preferentially binds to a mutant tumor suppressor protein.

18. (canceled)

19. The reporter conjugate of claim 10, wherein the reporter conjugate consists essentially of a single double-stranded targeting nucleic acid that binds specifically to a nucleic acid binding protein, the targeting nucleic acid being linked to one or more paramagnetic iron oxide particles.

20.-21. (canceled)


SEQUENCE LISTING (15 sequences)

SEQ ID NO: 1 - DNA, 7 bp, Artificial sequence
  Synthetic oligonucleotide
tgactca

SEQ ID NO: 2 - DNA, 8 bp, Artificial sequence
  Synthetic oligonucleotide
tgacgtca

SEQ ID NO: 3 - DNA, 6 bp, Artificial sequence
  Synthetic oligonucleotide
cccgcc

SEQ ID NO: 4 - DNA, 11 bp, Artificial sequence
  Synthetic oligonucleotide
ggggactttcc

SEQ ID NO: 5 - DNA, 20 bp, Artificial sequence
  Synthetic oligonucleotide
tccggctgactcatcaagcg

SEQ ID NO: 6 - DNA, 20 bp, Artificial sequence
  Synthetic oligonucleotide
aggccgactgagtagttcgc

SEQ ID NO: 7 - DNA, 19 bp, Artificial sequence
  Synthetic oligonucleotide
ctctctgacgtcaggcaat

SEQ ID NO: 8 - DNA, 19 bp, Artificial sequence
  Synthetic oligonucleotide
gagagactgctgtccgtta

SEQ ID NO: 9 - DNA, 20 bp, Artificial sequence
  Synthetic oligonucleotide
ctcgccccgccccgatcgaa

SEQ ID NO: 10 - DNA, 20 bp, Artificial sequence
  Synthetic oligonucleotide
gagcggggcggggctagctt

SEQ ID NO: 11 - DNA, 20 bp, Artificial sequence
  Synthetic oligonucleotide
gttgaggggactttcccagg

SEQ ID NO: 12 - DNA, 20 bp, Artificial sequence
  Synthetic oligonucleotide
caactcccctgaaagggtcc

SEQ ID NO: 13 - DNA, 26 bp, Artificial sequence
  Synthetic oligonucleotide
catcatggtcgtggtttgggcaaacc

SEQ ID NO: 14 - DNA, 26 bp, Artificial sequence
  Synthetic oligonucleotide
gagggagagcatagccctcgtagatg

SEQ ID NO: 15 - DNA, 22 bp, Artificial sequence
  Synthetic oligonucleotide
ccttagcggatgttgaccctgg